(19) United States
(12) Patent Application Publication
(10) Pub. No.: US 2009/0076515 A1
(43) Pub. Date: Mar. 19, 2009

(54) INSTRUMENT FOR DISIMPACTING A DAMAGED VERTEBRAL BODY

(75) Inventors: Claudio Lamartina, Milano (IT); Dirk Wiedmann, Schaffhausen (CH)

Correspondence Address: CROMPTON SEAGER AND TUFTE, LLC, 1221 NICOLLET AVENUE, SUITE 800, MINNEAPOLIS, MN, 55403-2420, US

(73) Assignee: ZIMMER GMBH, Winterthur (CH)

(21) Appl. No.: 11/911,251

(22) PCT Filed: Apr. 13, 2006
(86) PCT No.: PCT/EP2006/003426

§ 371 Date: Sep. 25, 2008

(30) Foreign Application Priority Data
Apr. 14, 2005 (EP) 05008186.8

(51) Int. Cl. A61B 17/58; A61B 17/00

(52) U.S. Cl. 606/90; 606/79

(57) ABSTRACT

The invention relates to an instrument for disimpacting a damaged vertebral body, comprising a unit, whose effective length can be adjusted in a longitudinal direction and whose underside can be positioned on the skin above a damaged vertebral body. According to the invention, supports that can be displaced in relation to one another are located on the adjustable unit, the distance between the exteriors of the two supports that face away from one another in the longitudinal direction defining the effective length of the adjustable unit. The adjustment range of the effective length is dimensioned in such a way that the supports can be positioned between lever elements, which are provided for the distraction of the vertebral bodies lying cranially and caudally adjacent to the damaged vertebral body and the supports are designed to maintain the mutual distance between the lever elements.

### FIELD OF THE INVENTION

The invention relates to an instrument and to a method for the disimpacting of a collapsed vertebral body.

### BACKGROUND OF THE INVENTION

A break of the vertebral body is a fracture of a vertebral body of the spinal column. A fracture of this type can, for example, be the consequence of an accident (traumatic fracture of a vertebral body) or it can be caused by osteoporosis. A plurality of methods are known from the prior art to disimpact a collapsed vertebral body again.

In vertebroplasty, a cannula is inserted into the patient, who lies on his stomach for the procedure, and indeed into the affected vertebral body, from the rear in a percutaneous transpedicle manner under X-ray monitoring. Bone cement is then injected through the cannula to disimpact and stabilize the vertebral body. In this method, the collapsed vertebral body is, however, usually not fully disimpacted. In vertebroplasty, it can furthermore result that a larger pressure is required on the bone cement and that as a consequence the injected bone cement can be discharged again via the fractured surfaces or the venous blood vessel system.

In kyphoplasty, a balloon catheter is inserted into the patient, who lies on his stomach for the procedure, and indeed into the affected vertebral body, from the rear via a cannula in a percutaneous transpedicle manner under X-ray monitoring. The balloon catheter is inflated by an X-ray contrast liquid to disimpact the vertebral body. After the contrast liquid has been drained, the balloon catheter is removed. Bone cement is then injected via the cannula into the hollow space left by the balloon catheter in order to stabilize the vertebral body. In this method, the collapsed vertebral body, onto which the two adjacent vertebral bodies press, is, however, usually likewise not fully disimpacted. The balloon catheters required for kyphoplasty are moreover expensive.

For this reason, so-called lordoplasty is recently being frequently used in addition to vertebroplasty and kyphoplasty. This is a method which is shown in FIG. 6 and in which lever elements 63, 67—cannulae in this example—are introduced, and in particular cemented, in the cranially and caudally adjacent vertebral bodies 73, 77 of the vertebral body 75 to be treated and are used as lever arms in order to relieve the collapsed vertebral body 75 before the injection of bone cement into it. The original level of the vertebral body and the original spinal curvature can hereby be at least approximately restored. A balloon catheter is not necessary for this purpose.

It can result with lorodplasty that the cannulae bend comparatively strongly relative to one another during levering, i.e. during compression, so that the desired lever effect can frequently not be achieved, at least not completely. For example, the cannulae can already have mutual contact without the desired relief for the collapsed vertebral body being reached. In this procedure, the involved vertebral bodies are moreover pressed onto one another at posterior and are thus exposed to an increased pressure there, whereby bone fragments can be pressed into the neural canal. Finally, the hands of the surgeon actuating the lever arms or the cannulae and holding them in position are exposed to high doses of radiation since the surgery takes place at least partly under X-ray monitoring.

An instrument and a method of the initially named kind should be set forth with which the methods described above can be improved.

### SUMMARY OF THE INVENTION

The instrument set forth in claim 1, the instrument system set forth in claim 18 as well as the method set forth in claim 27, in addition to a number of further advantageous properties, should satisfy this demand.

With the instrument set forth here, a device—also called a spacer in the following—is provided whose effective length can be adjusted in its longitudinal direction and whose lower side can be positioned on the skin over a collapsed vertebral body. Provision is further made that abutments adjustable relative to one another are arranged at the adjustable device, that the spacing of the outer sides of two abutments remote from one another in the longitudinal direction defines the effective length of the adjustable device, that the adjustment range of the effective length is dimensioned such that the abutments can be positioned between lever elements which are provided for the distraction of the vertebral bodies cranially and caudally adjacent to the collapsed vertebral body, and that the abutments are configured for the maintenance of the mutual spacing of the lever elements.

The lever elements can be cannula, for example biopsy needles. The lever elements are therefore also called cannulae in the following, with the properties and advantages set forth generally applying to any desired type of lever elements.

The abutments can be arranged adjacent to the lower side of the adjustable device. The abutments can furthermore project to the side of the adjustable device.

To ensure the longitudinal adjustability, the spacer can have a base element and an adjustment element adjustable relative to the base element in an embodiment, with at least one abutment being connected to the base element and at least one abutment being connected to the adjustment element. The effective length of the spacer is matchable to the mutual spacing of the cannulae, in particular at the points of entry into or exit from the skin. The spacer can in particular fix a mutual minimum distance between the cannulae.

The abutments provided for the fixing of the mutual spacing of the cannulae are also called spacing means in the following.

The spacing means of the base element can be associated both with the cranial cannula and with the caudal cannula. The spacing means of the adjustment element is then associated with the respectively other direction in accordance with the cannula.

The spacing means can project from the base element and the adjustment element to the left and/or to the right, in particular symmetrically from a center plane of the spacer.

A spacing means can in particular be provided for each of the base element and the adjustment element, said spacing means being made as an uninterrupted bar or the like and projecting to the left and to the right simultaneously.

However, two or more spacing means can also be provided for the base element and the adjustment element, of which at least one spacing means projects to the left and at least one spacing means projects to the right. Spacing means which project to the left and to the right are particularly suitable in those cases in which two respective lever elements, e.g. cannulae, are inserted for each adjacent vertebral body.

The matching to the mutual spacing of the cannulae in particular takes place with non-actuated cannulae, i.e. before the cranial cannula and the caudal cannula are compressed relative to one another. The mutual spacing of the cannulae, in particular at the skin or in the proximity of the skin, which is present with non-actuated cannulae, can also be maintained by the spacer with actuated cannulae, i.e. in a state in which the cannulae are compressed relative to one another. It can hereby be prevented that the involved vertebral bodies are pressed onto one another at posterior and are each exposed to an increased pressure there.

The instrument system set forth here includes an instrument of the type set forth here as well as lever elements, e.g. cannulae, which can be introduced into vertebral bodies and are provided for the distraction of the vertebral bodies cranially and caudally adjacent to a collapsed vertebral body.

A method in which the instrument or instrument system set forth here is used includes the introduction of at least one device for the introduction of bone cement into the collapsed vertebral body and in each case of at least cranial lever element and one caudal lever element into the vertebral bodies directly cranially and caudally adjacent to the collapsed vertebral body, the positioning of the instrument between the cranial lever element and the caudal lever element on the skin, whereby the mutual minimum spacing of the lever elements is fixed in the region of the abutments of the instruments, the pressing together of the lever elements relative to one another at regions projecting from the skin, whereby a distraction of the cranially and caudally adjacent vertebral bodies is effected and the collapsed vertebral body is relieved, and the introduction of bone cement into the collapsed vertebral body through the device for the introduction of bone cement.

It is possible to introduce two respective devices bodies for the introduction of bone cement or lever elements, e.g. cannulae, into the collapsed vertebral body and the directly adjacent vertebral.

Further embodiments are also set forth in the dependent claims, in the description as well as in the drawing, with the features of these embodiments being able to be combined with one another in any desired manner per se.

Provision can be made for the position of the adjustment element to be locked at the base element.

The base element can have at least one longitudinal member orientated in the longitudinal direction.

At least a part of the base element can be made in the manner of a frame which has two longitudinal members arranged as side parts. Material and weight can be saved by the at least part design of the base element as a frame. Furthermore, space can be provided for the adjustment element and/or for the adjustment mechanism to allow a particularly compact design of the instrument.

Elongate holes in which the adjustment element is adjustably supported can in particular be formed in the longitudinal members or side parts of the frame. The support of the adjustment element at the base element and simultaneously the longitudinal adjustability of the adjustment element relative to the base element is ensured, in particular in a particularly simple manner, by the elongate holes. Material and weight can additionally be saved by the elongate holes.

At least one of the elongate holes has a plurality of locking positions for the setting of different relative positions between the adjustment element and the base element. The spacer whose effective length is adapted to the spacing of the cannulae in particular at the entry positions into the skin can hereby be locked in the respective length. The locking positions can be made in the form of recesses shaped like an arc of a circle in the longitudinal sides of the elongate hole. An element of the adjustment element which is circular in cross-section at least in part can be supported in a fixed and immovable manner in a recess of this type.

Provision can furthermore be made that the adjustment element includes an adjustment part and an operating part which is connected to the adjustment part and which can be switched between a release position and a blocked position relative to the adjustment part, with the adjustment part and the operating part in particular being hingedly connected to one another. In the release position of the operating element, the spacer can be adjusted in its effective length along the adjustment device and can subsequently locked by moving into the blocked position with the adjusted length. With a hinged connection between the adjustment part and the operating part, it is made possible that the adjustment part can remain immobile in its position on the switching over of the operating part.

The operating part can project through a frame slot formed between longitudinal members in a frame of the base element. The operating part is thereby so-to-say encompassed by the frame so that a particularly compact design is made possible.

The in particular arcuate operating part can be connected rotationally rigidly to an axle of the adjustment element supported at the base element. The switch movement of the operating part between a release position and a blocked position can thus be a rotary movement. Since the axle is rotationally rigidly connected to the operating part, both the axle supported at the base part and the operating part are rotated on a switch movement of the operating part.

Furthermore, at least one abutment or one spacing means can be formed at the adjustment part, with the adjustment part being hingedly connected to an axle of the adjustment element which is supported at the base element and which in particular corresponds to the axle rotationally rigidly connected to the operating part. On a rotary movement of the operating part, and thus of the axle, the adjustment part can remain immobile in its position since the adjustment part is hingedly connected to the axle.

It is possible that the axle is adjustably supported in elongate holes, in particular in elongate holes of side parts or longitudinal members of a frame of the base element. The axle is adjustable along the elongate holes. The axle additionally allows at least a part of the adjustment element, such as in particular the operating part, to be rotatable relative to the base element. The axle can in particular be smoothed at two oppositely disposed sides, with provision being made that the diameter of the axle substantially corresponds to the diameter of arcuate recesses in the longitudinal sides of an elongate hole. The smoothed axle, and thus the adjustment element, can in particular be locked in a fixed position to the base element and/or the locking can be released.

Furthermore, a stop element can be associated with each abutment at the adjustable device and is arranged adjacent to the upper side of the adjustable device, with the stop elements being arranged further outside than the abutments in the longitudinal direction of the adjustable device at least in a locked position of an adjustment element of the adjustable device such that the spacing between two abutments is smaller in the longitudinal direction than that between two associated stop elements.

Provision can in particular be made for the base element to have support means which are in particular arranged at the same level as support means of the adjustment element. The support means are provided to support the handle parts mounted on the spacer, in particular when a fixing device for the fixing of the spacing between the handle parts has not yet been mounted.

The operating part can include at least one support limb which is arranged outside a frame of the base element and serves as a stop element. The support limb can be provided as a support point or support region for a handle part which can be placed onto the spacer and which will be explained in more detail at another point. The handle part will also be called a guide part in the following.

The adjustment part can be adjustable along a cut-out of the base element which extends at its lower side. This can be advantageous since the adjustment element is provided with a spacing means which can hereby be positioned close to the skin and can fix the spacing of the cannula directly at the point of entry into the skin. The adjustment part can hereby in particular form a part of the support surface of the spacer on the skin.

In the instrument system set forth here, a respective handle part or guide part can be provided for each of a cranial and a caudal lever element and can be placed onto the adjustable device such that the guide part provides a contact surface for the respective lever element.

The regions of the lever elements, e.g. cannulae, projecting out of the body are thus also pressed together relative to one another by compressing the guide parts or handle parts relative to one another. Since the lever elements can contact the handle parts over a relatively large part region of their length, an excessive bending of the regions of the lever elements projecting out of the body can be effectively prevented.

The guide parts can each be made in the form of vanes.

To provide the contact surfaces, the handle parts can each have a plate-like base section which is in particular slit centrally for the placing on the spacer and/or is provided with lateral contact boundaries for the lateral guidance of the lever element. The central slot allows the base section also to be able to extend to the left and to the right of the spacer in the mounted state of the handle part so that a contact surface is available to the lever element over a part region which is as large as possible. The lateral contact boundaries prevent the lever element from giving way to the left or to the right on levering to escape the pressure of the handle part or of the base section.

The handle parts can furthermore each have an actuation section which is in particular S-shaped, with the actuation sections having a larger spacing from one another in the mounted state than base sections of the handle parts. The handle parts can be actuated via the actuation sections. To provide a sufficient leverage path and/or space for handle parts of the lever elements, e.g. cannulae, the spacing of the actuation sections can be larger than the spacing of the base sections.

In accordance with an embodiment, the handle parts can be connectable to one another via an adjustable fixing device, in particular an adjustable screw and/or a nut. The spacing can be fixed between the handle parts by the fixing device in the mounted state. This also applies in particular to a mounted state in which the two handle parts have already been pressed together relative to one another. The surgeon is thus not forced to hold the actuated handle parts or cannulae in position by hand e.g. during the injection of bone cement into the collapsed vertebral body so that the radiation exposure for the surgeon can be minimized. It is also possible for the fixing device to be used not only for the fixing of the two handle parts, but additionally for the pressing together of the two handle parts relative to one another per se.

To allow a simple assembly of the fixing device, at least one handle part can have an introductory slot through which the fixing device can be introduced into the handle part. The adjustable screw, in particular together with a nut, can hereby be pivoted so-to-say into the handle part relative to it. The introductory slot can be formed partly in a base section and partly in an actuation section of the handle part, with the part of the introductory slot formed in the actuation section allowing the passing through of the nut.

The method set forth here can include—after the positioning of the instrument on the skin and before the pressing together of the lever elements—setting the effective length of the instrument to the spacing between a cranial and a caudal lever element in the region of the exit point from the skin by displacement of an adjustment element relative to a base element and switching over an operating part of the adjustment element from a release position into a locked position relative to an adjustment part of the adjustment element.

The method can furthermore include—after the positioning of the instrument on the skin—introducing a respective guide element cranially and caudally between the device for the introduction of bone cement and a stop element associated with the abutment of the instrument and placing he guide parts onto the adjustable device of the instrument such that the respective guide part provides a contact surface for the lever element.

The method can furthermore include pressing together the guide parts relative to one another to press the regions of the lever elements projecting from the skin together relative to one another.

The method can furthermore include—after the pressing together of the lever elements—fixing the spacing between the guide parts by means of an adjustable fixing device.

The method can furthermore include providing devices for the introduction of bone cement as lever elements and—before the pressing together—introducing bone cement into the vertebral bodies directly cranially and caudally adjacent through the corresponding devices for the introduction of bone cement.

The introduction of bone cement can take place by injection.

### BRIEF DESCRIPTION OF THE DRAWINGS

The invention will be described in the following by way of example with reference to the enclosed drawing. There are shown:

FIG. 1 a perspective view of a spacer of an instrument set forth here;

FIG. 2 perspective views of components of the spacer (adjustable device) shown in FIG. 1;

FIG. 3 perspective views of handle parts (guide parts) of an instrument set forth here;

FIG. 4 different views of an instrument set forth here with the spacer (adjustable device) of FIG. 1 and the handle parts (guide parts) of FIG. 3;

FIG. 5 a further embodiment of an instrument set forth here, with cannulae (lever elements) additionally being shown which are inserted into the collapsed vertebral body and the directly adjacent vertebral bodies; and

FIG. 6 a representation of a surgical procedure known from the prior art.

### DETAILED DESCRIPTION OF THE INVENTION

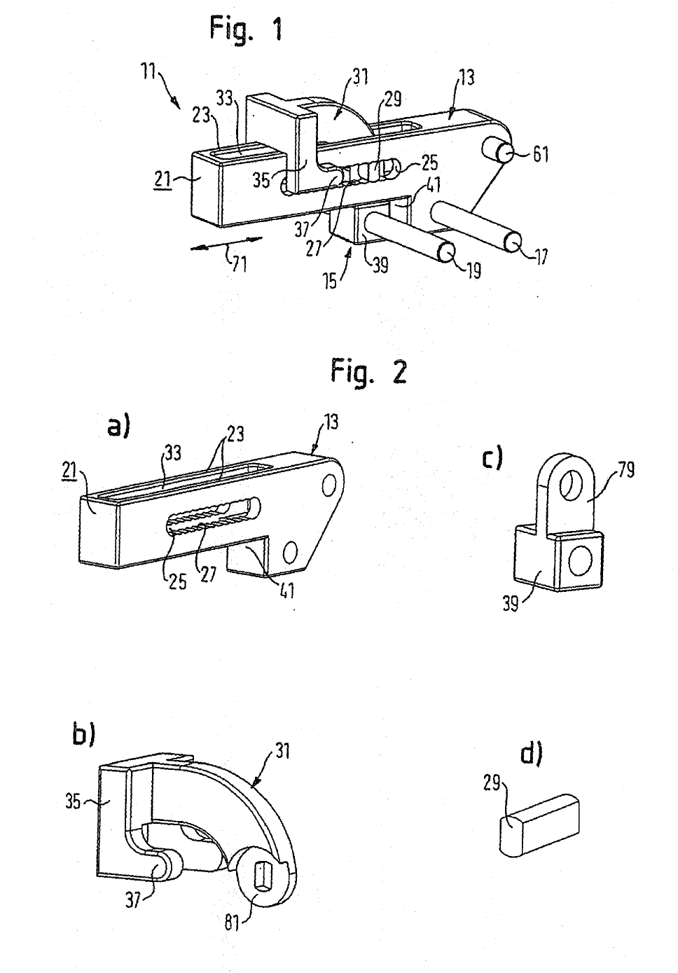

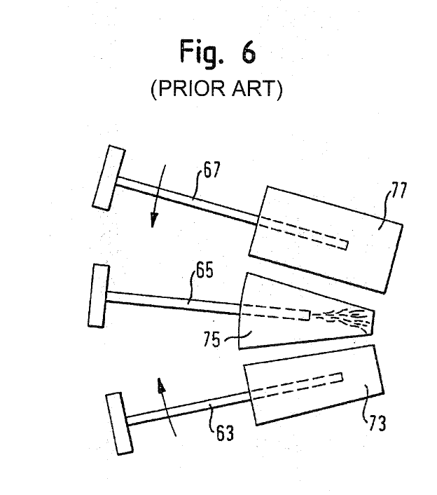

FIG. 1 shows a spacer 11 of an instrument or of an instrument system for the disimpacting of a collapsed vertebral body 75 which is only shown in connection with a surgical procedure known from the prior art in FIG. 6. The spacer includes a base element 13 which is additionally shown in FIG. 2a and a multipart adjustment element 15 whose individual parts 29, 31, 39 are additionally shown in FIGS. 2c to 2d. The adjustment element 15 is adjustable along an adjustment direction 71 at the base element 13 to set the effective length between a bar-shaped spacer shaft (abutment) 17 attached to a head section of the base element 13 and a likewise bar-shaped spacer shaft (abutment) 19 attached to the adjustment element 15.

The spacer shaft 17 and the spacer shaft 19 each extend perpendicular to the adjustment direction 71 of the adjustment element 15 or to the longitudinal axis of the spacer 11, are each guided through a cylindrical bore formed in the base element 13 or in the adjustment element 15 and project from the base element 13 or from the adjustment element 15 at both sides, in particular symmetrically, to a central plane of the spacer 11.

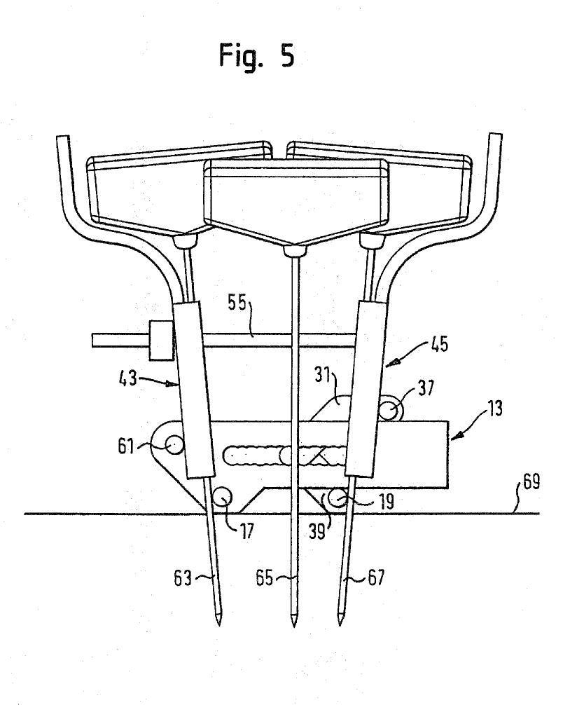

The spacer 11 is positionable between cannulae (lever elements) 63, 67, which are shown only in connection with a further embodiment of an apparatus in FIG. 5 and in FIG. 6, on the skin 69 (FIG. 5) of a patient who is lying on his stomach during the operation. The cannulae 63 and 67 are inserted into the directly adjacent vertebral bodies 73, 77 (FIG. 6) of the collapsed vertebral body 75. In FIGS. 5 and 6, two respective cannulae 63, 65, 67 are inserted into each of the adjacent vertebral bodies 73, 77 and into the collapsed vertebral body 75 to the left and to the right of the symmetrical plane of the vertebral bodies 73, 75, 77. The effective length of the spacer 11 is set such that the spacer shafts 17,19 inwardly contact the cannulae 63, 67 to fix the mutual spacing of the cannulae 63, 67 at the points of entry into the skin 69.

The base element 13 includes—in addition to the head section—a section made in the manner of a frame 21. A respective elongate hole 25 is formed in the two side parts 23 of the frame 21 and an axle 29 smoothed at two mutually opposite sides is adjustably supported in them. Each of the two elongate holes 25 has a plurality of locking positions for the adjustment of different relative positions between the adjustment element 15 and the base element 13 and are made in the form of arcuate recesses 27 in the longitudinal sides of the elongate hole 25.

The adjustment element 15 includes an adjustment part 39 with a cylindrical bore through which the spacer shaft 19 is pushed. The adjustment part 39 furthermore has a hinged section 79 which likewise has a cylindrical bore and through which the axle 29 extends in the assembled state of the adjustment element 15 in accordance with FIG. 1. The adjustment part 39 and the axle 29 are hingedly connected to one another, i.e. the axle 29 is freely rotatable inside the cylinder bore of the hinged section 79 of the adjustment part 39. The adjustment part 39, in particular the section 79 at which the spacer shaft 19 is formed, is adjustable along a cut-out 41 at the lower side of the base element 13. The spacer 11 thus lies above the fixed head section and the adjustable adjustment part 39 on the skin.

The adjustment element 15 furthermore includes an operating part 31. The operating part 31 is arcuate in shape and projects through a frame slot 33 formed in the frame 21 of the base element 13. The operating part 31 likewise includes a hinged section 81 in which a bore is formed for the axle 29 and which is arranged in the assembled state of the adjustment element 15 in accordance with FIG. 1 next to and contacting the hinged section 79 of the adjustment part 39. The contour of the bore of the hinged section 81 of the base element 13 is matched to the contour of the smoothed axle 29 such that the operating part 31 is rotationally rigidly connected to the axle 29.

The operating part 31 is switchable between a blocked position, in which the adjustment element 15 is immovably locked to the base element 13 and which is shown in FIG. 1, and a release position, in which the adjustment element 71 is adjustable relative to the base element 13. In the locked position, the non-smoothed sides of the axle 29 engage in a shaped-matched and force-transmitting manner into the arcuate recesses 27 in the longitudinal sides of the elongate holes 25 so that an adjustability along the adjustment direction 71 is effectively suppressed. This is in particular achieved in that the diameter of the axle 29 substantially corresponds to the diameter of the arcuate recesses 27 in the longitudinal sides of the elongate holes 25.

The release position is reached in that the operating part 31 is pivoted about the axle 29, with the axle 29 also turning along due to the rotationally rigid connection between the operating part 31 and the axle 29. This has the result that the non-smoothed sides of the axle 29 and the recesses 27 in the longitudinal sides of the elongate holes 25 are brought out of engagement so that the adjustment element 15 can be adjusted relative to the base element 13 in the direction of adjustment 71.

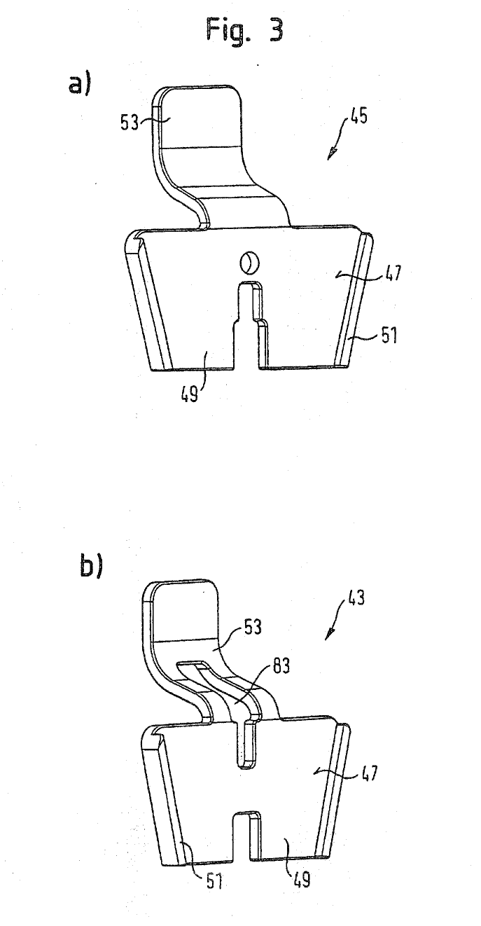

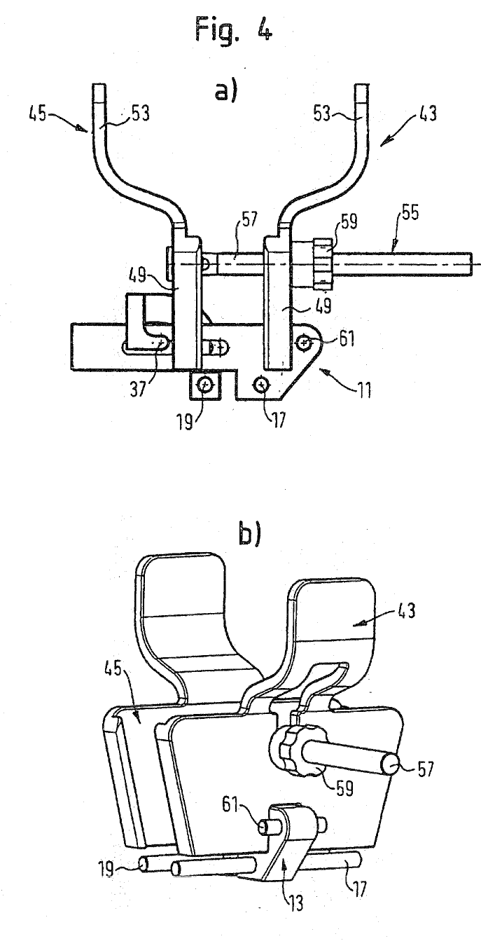

The operating part 31 has a respective support limb 35 both at the left and at the right at its end which projects upwardly out of the frame slot 33 of the base element 13, said support limb extending to the side of the side parts 23 of the frame 21 outside the frame 21 of the base element 13. A respective projection (stop element) 37 is formed at the support limbs 35 which is directed in the adjustment direction 71 and, analogously to a support shaft (stop element) 61 which is secured to the head section of the base element 13 via a cylinder bore and projects from the spacer 11 symmetrically to the left and to the right, serves as a support means for handle parts (guide parts) 43, 45 shown in FIG. 3.

The handle parts 43, 45, which are each made in the manner of a vane, can be mounted cranially and caudally onto the spacer 11 such that the handle parts 43, 45 provide a contact surface 47 for the cranial and caudal cannulae 63, 67. The handle parts 43, 45 each have a plate-like base section 49 which is slit centrally for the placing onto the spacer 11. The base section 49 is moreover provided with lateral contact boundaries 51 to provide a guidance for the cannulae 63, 67 so that they cannot give way to the left or to the right on an actuation of the handle parts 43, 45. The handle parts 43, 45 furthermore each have S-shaped actuation sections 53 which directly adjoin the base sections 49. In the state mounted onto the spacer 11 (FIG. 4), the actuation sections 53 have a larger spacing from one another than the base sections 49.

Finally, the instrument for the disimpacting of a collapsed vertebral body 75 includes an adjustable fixing device 55 which is shown in FIG. 4 and via which the two handle parts 43, 45 are connected to one another in the state mounted onto the spacer 11. The fixing device 55 includes an adjustable screw 57 which is guided into or through a circular opening formed in the handle part 45 and an introductory slot 83 formed in the handle part 43, with a nut 59 being screwed onto the end the adjustable screw 57 projecting out of the introductory slot 83 to fix the mutual position of the two handle parts 43, 45.

A further, second embodiment of an instrument for the disimpacting of a collapsed vertebral body 75 is shown in FIG. 5, with the same or corresponding parts each being designated by the same reference numerals so that only the differences of the second embodiment with respect to the first embodiment shown in FIGS. 1 to 4 are explained.

In the apparatus in accordance with FIG. 5, a support shaft 37, which projects to the left and to the right from the spacer 11, is attached to the upper end of the operating part 31. The support shaft 37 takes over the function of the projections 37 of the first embodiment. Ultimately, the second embodiment only differs from the first embodiment in that the support elements 61 associated with the base element 13 are not arranged at the same level as the support means 37 associated with the adjustment element 15.

A possible method for the disimpacting of a collapsed vertebral body will be described in the following with reference to the Figures.

First, two respective cannulae 63, 65, 67 are inserted into the collapsed vertebral body 75 and the directly adjacent vertebral bodies 73, 77. Bone cement is then injected into the directly adjacent vertebral bodies 73, 77. As soon as the bone cement injected into the adjacent vertebral bodies 73, 77 has hardened, the surgical procedure can be continued. It is naturally also possible for the cannulae 65 provided for the collapsed vertebral body 75 only to be inserted after the insertion of the bone cement into the directly adjacent vertebral bodies 73, 77 and/or to be inserted after the hardening of the bone cement in the adjacent vertebral bodies 73, 77.

Next, the spacer 11 adjustable in its effective length is positioned on the skin 69 of a patient such that the mutual spacing of the cranial and caudal cannulae 63, 67 is fixed to the points of entry into the skin. For this purpose, the effective length of the spacer 11 is set in the release position such that the spacer shaft 17 and the spacer shaft 19 each contact the inner sides of the cranial and caudal cannulae 63, 67 respectively. Once this has been achieved, the operating part 31 is switched from the release position into the blocked position to lock the length of the spacer 11 at the corresponding spacing. It is naturally also possible for the insertion of the cannulae 65 into the collapsed vertebral body 75 for the injection of the bone cement into the directly adjacent vertebral bodies 73, 77 and/or for the hardening of the bone cement in the adjacent vertebral bodies 73, 77 to be carried out during or after the positioning or the length adjustment of the spacer 11.

The two handle parts 43, 45 are subsequently introduced between the cranial and caudal cannulae 63, 67 and the spacer shafts 17, 19 of the spacer 11 and are mounted onto the spacer 11 such that the handle parts 43, 45 provide a contact surface 47 for the cannulae 63, 67. The following sequence is thus present (FIG. 5) when considered in the adjustment direction 71—in each case from the outside to the inside: Support shaft 61 or 37—handle part 43 or 45—cannula 63 or 67—spacer shaft 17 or 19.

The handle parts 43, 45 are then pressed together relative to one another such that the regions of the cannulae 63, 67 projecting out of the body of the patient are likewise pressed together relative to one another. It is hereby achieved that the fractured vertebral body 75 is relieved. The two spacer shafts (abutments) 17 and 19 act as pivot points or “kink points” for the cranial and caudal cannulae 63, 67. The regions of the cannulae 63, 67 acted on by the base sections 49 of the handle parts 43, 45 are not bent in this process.

As soon as the desired spacing between the handle parts 43, 45 has been reached which corresponds to a desired relief of the collapsed vertebral body 75, the spacing between the handle parts 43, 45 is fixed by means of the fixing device 55. This is in particular achieved in that the nut 59 is tightened up to abutment with the handle 43. It is generally also possible for the fixing device 55 to be used for the pressing together of the two handle parts 43, 45 relative to one another per se.

Finally, bone cement is injected into the collapsed vertebral body 75 via the cannulae 65 to stabilize the collapsed vertebral body 75. After the hardening of the bone cement injected into the collapsed vertebral body 75, the instrument and the cannulae 63, 65, 67 inserted into the affected vertebral bodies 73, 75, 77 can be removed. The surgical procedure takes place at least partly under X-ray monitoring.

The instrument set forth here for the disimpacting of a collapsed vertebral body 75, which at least includes the spacer 11 explained above and in particular also the two handle parts 43, 45 explained above and the fixing device 55, makes it possible for all previously known surgical techniques described in connection with the prior art to be able to be improved. In particular a better treatment of collapsed vertebral bodies can be ensured by the instrument and/or method set forth here.

What is claimed is:

1. An instrument for disimpacting a collapsed vertebral body, comprising a device whose effective length can be adjusted in a longitudinal direction and whose lower side can be positioned on the skin above a collapsed vertebral body,
wherein abutments which are adjustable relative to one another are arranged at the adjustable device;
wherein the spacing of the mutually remote outer sides of two abutments in the longitudinal direction defines the effective length of the adjustable device;
wherein the adjustment range of the effective length is dimensioned such that the abutments can be positioned between lever elements which are provided for the distraction of the vertebral bodies cranially and caudally adjacent to the collapsed vertebral body; and
wherein the abutments are configured for the maintenance of the mutual spacing of the lever elements and are each made as the fulcrum for the respective lever element at their mutually remote outer sides.

2. An instrument in accordance with claim 1, characterized in that the abutments are arranged adjacent to the lower side of the adjustable device.

3. An instrument in accordance with claim 1, characterized in that the abutments project laterally from the adjustable device.

4. An instrument in accordance with claim 1, characterized in that the adjustable device has a base element and an adjustment element adjustable relevant to the base element, with at least one abutment being connected to the base element and at least one abutment being connected to the adjustment element.

5. An instrument in accordance with claim 4, characterized in that the position of the adjustment element at the base element can be locked.

6. (canceled)

7. An instrument in accordance with claim 4, characterized in that at least one part of the base element is configured in the manner of a frame which has two longitudinal members arranged as side parts.

8. An instrument in accordance with claim 7, characterized in that elongate holes in which the adjustment element is adjustably supported are made in the longitudinal members.

9. An instrument in accordance with claim 8, characterized in that at least one of the elongate holes has a plurality of locking positions for the adjustment of different relative positions between the adjustment element and the base element, with the locking positions in particular being made in the form of arcuate recesses in the longitudinal sides of the elongate hole.

10. An instrument in accordance with claim 4, characterized in that the adjustment member comprises an adjustment part and an operating part which is connected to the adjustment part and which can be switched relative to the adjustment part between a release position and a blocked position, with the adjustment part and the operating part in particular being hingedly connected to one another.

11. An instrument in accordance with claim 10, characterized in that the operating part projects through a frame slot formed between the longitudinal members in a frame of the base element.

12. An instrument in accordance with claim 10, characterized in that the in particular arcuate operating part is connected rotationally rigidly to an axle of the adjustment element supported at the base element.

13. An instrument in accordance with claim 10, characterized in that at least one abutment is formed at the adjustment part, with the adjustment part being hingedly connected to an axle of the adjustment element which is supported at the base element.

14. An instrument in accordance with claim 12, characterized in that the axle in particular smoothed at two oppositely disposed sides is adjustably supported in elongate holes of the base element, with the diameter of the axle in particular substantially corresponding to the diameter of arcuate recesses in the longitudinal sides of an elongate hole.

15. An instrument in accordance with claim 1, characterized in that a stop element is associated with each abutment at the adjustable device and is arranged adjacent to the upper side of the adjustable device, with the stop elements being arranged further outside than the abutments in the longitudinal direction of the adjustable device at least in a locked position of an adjustment element of the adjustable device such that the spacing between two abutments is smaller in the longitudinal direction than that between two associated stop elements.

16. (canceled)

17. An instrument in accordance with claim 10, characterized in that the adjustment part is adjustable along a cut-out of the base element which extends at its lower side.

18. An instrument system, comprising an instrument in accordance with claim 1 as well as lever elements which can be introduced into the vertebral bodies and are provided for the distraction of the vertebral bodies cranially and caudally adjacent to a collapsed vertebral body.

19. An instrument system in accordance with claim 18, characterized in that a respective guide part is provided for a cranial and a caudal lever element and can be placed onto the adjustable device such that the guide part provides a contact surface for the respective lever element.

20. (canceled)

21. An instrument system in accordance with claim 19, characterized in that the guide parts each have a plate-like base section which is in particular slit centrally for the placing onto the adjustable device and/or is provided with lateral stop boundaries for the lateral guidance of the lever element.

22. An instrument system in accordance with claim 19, characterized in that the guide parts each have an actuation section which is in particular S-shaped, with the actuation sections having a larger spacing from one another in the placed on state than base sections of the guide parts.

23. (canceled)

24. (canceled)

25. An instrument system in accordance with claim 18, characterized in that the lever elements are likewise made as devices for the introduction of bone cement.

26. (canceled)

27. A method of disimpacting a collapsed vertebral body, comprising
introducing at least one device for the introduction of bone cement into the collapsed vertebral body and in each case at least one cranial and one caudal lever element into the vertebral bodies directly cranially and caudally adjacent to the collapsed vertebral body;
positioning an instrument in accordance with claim 1 between the cranial lever element and the caudal lever element on the skin, whereby the mutual minimum spacing of the lever elements is fixed in the region of the abutments of the instrument;
pressing together the lever elements relative to one another at regions projecting from the skin, whereby a distraction of the cranially and caudally adjacent vertebral body is effected and the collapsed vertebral body is relieved; and
introducing bone cement into the collapsed vertebral body through the device for the introduction of bone cement.

28. A method in accordance with claim 27, comprising,
after the positioning of the instrument on the skin and before the pressing together of the lever elements,
setting the effective length of the instrument to the spacing between a cranial and a caudal lever element in the region of the exit point from the skin by displacement of an adjustment element relative to a base element; and
switching over an operating part to the adjustment element from a release position into a locked position relative to an adjustment part of the adjustment element.

29. A method in accordance with claim 27, comprising,
after the positioning of the instrument on the skin, introducing, cranially and caudally, a respective guide part between the device for the introduction of bone cement and a stop element associated with the abutment of the instrument; and
placing the guide parts onto the adjustable device of the instrument such that the respective guide part provides a contact surface for the lever element.

30. A method in accordance with claim 29, comprising
pressing together the guide parts relative to one another to press together the regions of the lever elements projecting from the skin relative to one another.

31. (canceled)

32. A method in accordance with claim 27, comprising
providing devices for the introduction of bone cement as lever elements and, before the pressing together,
introducing bone cement into the directly cranially and caudally adjacent vertebral bodies through the corresponding devices for the introduction of bone cement.

33. (canceled)
